(19) United States
(12) Patent Application Publication
(10) Pub. No.: US 2026/0008034 A1
(43) Pub. Date: Jan. 8, 2026

(54) LOW-CARBON ALKANE DEHYDROGENATION CATALYST, PREPARATION METHOD THEREFOR AND APPLICATION THEREOF

(71) Applicant: REZEL ENGINEERING CO., LTD, Shanghai (CN)

(72) Inventors: Ruiling ZHAO, Shanghai (CN); Runsheng ZHUO, Shanghai (CN); Qiushi SUN, Shanghai (CN); Bing LIU, Shanghai (CN); Xingyue LAN, Shanghai (CN); Chunxue ZHANG, Shanghai (CN); Yusen RAO, Shanghai (CN); Xinsheng LIU, Shanghai (CN)

(21) Appl. No.: 18/992,137

(22) PCT Filed: Jul. 6, 2023
(86) PCT No.: PCT/CN2023/106191

§ 371 Date: Jan. 7, 2025

(30) Foreign Application Priority Data
Jul. 7, 2022 (CN) 202210804288.2

(51) Int. Cl. B01J 23/62; B01J 6/00; B01J 23/10; B01J 23/656; B01J 35/61; B01J 35/63; B01J 35/64; B01J 37/02; B01J 37/03; B01J 37/08; C07C 5/32; C07C 9/08; C07C 9/12

(52) U.S. Cl. CPC B01J 23/62; B01J 6/001; B01J 23/10; B01J 23/626; B01J 23/6562; B01J 35/613; B01J 35/638; B01J 35/647; B01J 37/0201; B01J 37/036; B01J 37/08; C07C 5/325; C07C 9/08; C07C 9/12

(57) ABSTRACT

The present invention provides a light alkane dehydrogenation catalyst, and a preparation method and application thereof, and belongs to the technical field of petrochemical technology. The catalyst uses at least one of precious metals Pt, Pd, Ru and Rh as an active component, at least one of transition metals Ga, V, In, Sn, Mn, Ce, Fe and Ni as a promoter, and a modified zinc aluminate carrier as a carrier; the chemical composition of the modified zinc aluminate carrier is of the general formula ZnMₓAl_{y}O₄, where x is 0.01-0.99, y is 0.01-1.99, and it satisfies x+y=2; M is selected from at least one of the rare earth elements La, Ce, Pr, Sm and Er. The catalyst prepared by such a modified zinc aluminate carrier has the characteristics of high propane conversion rate, high selectivity for product propylene, strong resistance to sintering, good stability, etc.

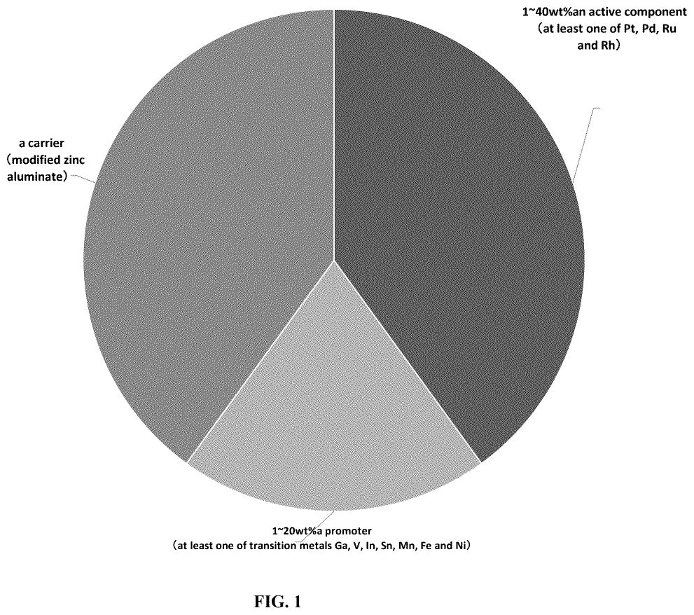

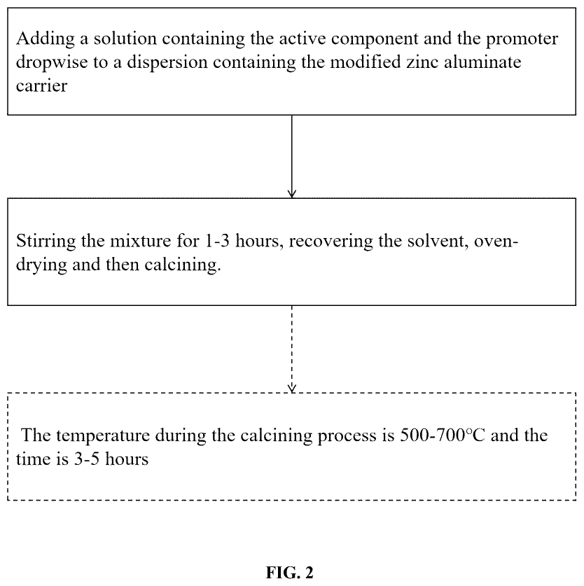

### TECHNICAL FIELD

The present invention belongs to the technical field of petrochemical technology, and specifically related to a light alkane dehydrogenation catalyst, and a preparation method and application thereof.

### BACKGROUND

Propylene, as a raw material for the production of chemical products such as polypropylene, acrylonitrile, and propylene oxide, etc., is an important organic basic chemical raw material with a yield second only to ethylene. As of the end of 2019, China's annual propylene production was 32.88 million tons. At present, the main sources of propylene supply are steam cracking of naphtha and catalytic cracking processes, both of which are considered as oil-to-propylene production routes. However, the technology of traditional propylene production route is difficult to meet the market demand. Therefore, it is particularly important to develop new oriented and efficient propylene production technologies.

Propane direct dehydrogenation is one of the oriented production processes for propylene in industry. Compared with traditional oil to propylene production routes, this process has the advantages of high selectivity to propylene, abundant raw material sources, simple product composition, and ease of separation, etc. At present, the propane anaerobic dehydrogenation process has been successfully industrialized, mainly including Catofin process from Lummus Company, Oleflex process from UOP Company, and STAR process from Uhde Company. Among them, Catofin and Oleflex processes are the most widely used. The Catofin process uses CrOₓ/Al₂O₃ as the catalyst and employs a fixed bed reactor, while the Oleflex process uses Pt/Al₂O₃ as the catalyst and employs a fluidized bed reactor.

Compared with CrOₓ/Al₂O₃, Pt-based catalysts have the advantages of high reactivity, high selectivity to propylene, and low toxicity, etc., and are favored by people. However, Pt particles are prone to sintering at high temperatures, and their stability is poor and they are prone to deactivation due to the accumulation of carbon deposition on the surface, greatly reducing the production capacity of the equipment. Therefore, it is particularly important to develop Pt-based catalysts with high stability and strong resistance to carbon deposition.

At present, there are many studies on Pt-based catalysts by scholars at home and abroad. Chinese patent CN109746033B uses a molecular sieve with a special structure as the carrier and PtSn as the active component to prepare a dehydrogenation catalyst with a special structure. However, the patent does not provide the stability data and resistance to carbon deposition of the catalyst. Chinese patent CN102247843A discloses a method for improving the stability of Pt-based catalysts for the dehydrogenation of cycloalkanes. The method involves adding oxide active components CaO, ZrO₂, BaO, La₂O₃, and CeO₂ to the carrier of Pt/Al₂O₃ catalyst, the improved catalyst is used for the dehydrogenation of cyclohexane, a hydrogen storage material, and the stability of the catalyst is improved. However, the initial dehydrogenation performance of the catalyst provided in this patent is poor.

In summary, the zinc aluminate carrier based dehydrogenation catalysts reported in the existing inventions have low catalytic activity, low selectivity, and weak resistance to carbon deposition, therefore their longitudinal and transverse properties still need to be further improved.

### Contents of Invention

The object of the present invention is to provide a light alkane dehydrogenation catalyst, and a preparation method thereof. The catalyst prepared by such a modified zinc aluminate carrier has the characteristics of high propane conversion rate, high selectivity to product propylene, strong resistance to sintering, good stability, etc.

The present invention is implemented by the following technical solutions:

In a first aspect, the present invention provides a light alkane dehydrogenation catalyst, the catalyst uses at least one of precious metals Pt, Pd, Ru and Rh as an active component, at least one of transition metals Ga, V, In, Sn, Mn, Ce, Fe and Ni as a promoter, and the modified zinc aluminate carrier as ta carrier;

The modified zinc aluminate carrier has a chemical composition of the general formula ZnMₓAl_{y}O₄, where x is 0.01-0.99, y is 0.01-1.99, and it satisfies x+y=2; M is selected from at least one of the rare earth elements La, Ce, Pr, Sm and Er.

Further, in a preferred embodiment of the present invention, based on the total mass of the catalyst on a dry basis, the content by mass percentage of the active component is 1-40 wt %, the content by mass percentage of the promoter is 1-20 wt %, with the balance being the modified zinc aluminate carrier.

Further, in a preferred embodiment of the present invention, the specific surface area of the modified zinc aluminate carrier is 10-100 m²/g, the pore size range is 3 nm-30 nm, and the pore volume range is 0.1-0.7 g/mL.

Further, in a preferred embodiment of the present invention, a precursor of the rare earth element is one or more of nitrate of the rare earth element, rare earth metal oxide, rare earth metal sulfate and rare earth metal organic acid salt.

Further, in a preferred embodiment of the present invention, a precursor of the precious metal element is selected from one or more of metal halides, metal nitrates and metal complexes;

preferably, a precursor of the transition element is one or more of oxides, inorganic salts and complexes of a metal element.

Further, in a preferred embodiment of the present invention, the modified zinc aluminate carrier is prepared by a gel sol method, an impregnation method, a precipitation method, a coprecipitation method or a hydrothermal synthesis method.

Further, in a preferred embodiment of the present invention, the modified zinc aluminate carrier is prepared by a precipitation method or a coprecipitation method, and the precipitant used is at least one of ammonia water, sodium hydroxide, potassium hydroxide, sodium carbonate and urea;

preferably, the modified zinc aluminate carrier is prepared by the gel sol method, and the gelling agent used is at least one of citric acid, nitric acid and hydrochloric acid.

In a second aspect, the present invention provides a preparation method of the above-mentioned light alkane dehydrogenation catalyst, comprising:

under stirring conditions, adding a solution containing an active component and a promoter dropwise to a dispersion containing a modified zinc aluminate carrier, stirring the mixture for 1-3 hours, recovering the solvent, oven-drying and then calcining.

Further, in a preferred embodiment of the present invention, the temperature during the calcining process is 500-700° C. and the time is 3-5 hours.

In a third aspect, the present invention provides application of the above-mentioned light alkane dehydrogenation catalyst, the catalyst is used for propane dehydrogenation, isobutane dehydrogenation, or propane/isobutane mixed gas dehydrogenation, and the catalyst is applied to a fixed bed, a moving bed or a fluidized bed, with a reaction temperature of 550-620° C., a reaction pressure of 10-150 kPa, and a reaction space velocity of 0.1-2 h⁻¹.

Compared with the prior art, the present invention at least has the following technical effects.

The catalyst provided by the present invention uses a special modified zinc aluminate carrier modified with a rare earth element as the carrier of the dehydrogenation catalyst, which improves the stability of the carrier and reduces the surface acidity of the carrier, thereby reducing the problem of acid cracking due to excessive B acid in the traditional carriers. Compared to modification of zinc aluminate with alkali metal or alkaline earth metal elements, modification of zinc aluminate with rare earth elements La, Ce, Pr, Sm and Er, such a modified zinc aluminate carrier in this application uses rare earth elements to be introduced into the lattice of the carrier to enhance the mechanical strength of the carrier structure. At the same time, the rare earth elements have strong electron storage and release capabilities, greatly regulating the existence state of active components on the surface of the carrier and the valence state during the reaction process.

By loading precious metals as the active components and introducing promoter metal elements, the valence state and electron cloud density of the active components during the reaction process can be adjusted to regulate its existence state on the surface of the carrier, greatly improving the conversion rate of light alkanes, inhibiting the deep dehydrogenation of alkanes to generate carbon deposition species, improving the thermal stability of the catalyst, and effectively suppressing the formation of carbon deposition. The light alkane dehydrogenation catalyst provided by the present invention has much better catalytic performance and catalyst stability than the existing industrial catalysts, and has potential industrial application prospects.

### DETAILED DESCRIPTION OF THE EMBODIMENTS

The implementation methods of the present invention will be described in detail below in conjunction with examples. However, those skilled in the art will understand that the following examples are only used to illustrate the present invention and should not be regarded as limiting the scope of the present invention. The specific conditions not specified in the examples are carried out according to conventional conditions or conditions recommended by the manufacturer. The reagents or instruments used without indicating the manufacturer are all conventional products that can be available commercially.

The technical solution of the present invention is as follows.

This embodiment provides a light alkane dehydrogenation catalyst, which can be used for propane dehydrogenation, isobutane dehydrogenation, or propane/isobutane mixed gas dehydrogenation. This catalyst is composed of an active component, a promoter and a carrier. Specifically:

### (1) Active Components

This catalyst uses at least one of the precious metals Pt, Pd, Ru and Rh as the active component, which mainly plays the role of breaking C—H bonds. Preferably, the catalyst uses any one of the precious metals Pt, Pd, Ru and Rh as the active component, and more preferably Pt as the active component. A precursor of the precious metal element is selected from one or more of metal halides, metal nitrates and metal complexes.

Among them, based on the total mass of the catalyst on a dry basis, the content by mass percentage of the active component is 1-40 wt %; preferably, the content by mass percentage of the active component is 5-35 wt %, and more preferably 10-25%. Controlling the content by mass percentage of the precious metal elements in the catalyst within 1%-40% is beneficial for the breakage of C—H bonds during the propane dehydrogenation reaction, beyond this range, adverse effect such as deep cracking may occur.

### (2) Promoter

This catalyst uses at least one of the transition metals Ga, V, In, Sn, Mn, Ce, Fe and Ni as a promoter, which mainly functions to change the valence state and electron cloud density of the active component during the reaction process to regulate its existence state on the surface of the carrier. Preferably, the promoter is any one of the transition metals Ga, V, In, Sn, Mn, Fe and Ni, and more preferably, the promoter is Ce, Fe, Mn, Sn, Ga. A precursor of the transition element is one or more of oxides, inorganic salts, and complexes of a metal element.

Wherein, based on the total mass of the catalyst on a dry basis, the content by mass percentage of the promoter is 1-20 wt %, preferably the content by mass percentage is 5-15 wt %, and more preferably the content by mass percentage is 8-12 wt %. Controlling the content by mass percentage of the promoter in the catalyst within 1-20% is beneficial for its regulatory effect on active components; and beyond this range, the active sites may be covered, resulting in adverse effects such as decreased catalytic activity, etc.

### (3) Carrier

The catalyst is supported on a modified zinc aluminate carrier, which mainly plays the role of dispersing and supporting the active components.

This modified zinc aluminate carrier has a chemical composition of the general formula ZnMₓAl_{y}O₄, where x is 0.01-0.99, y is 0.01-1.99, and it satisfies x+y=2; M is selected from at least one of the rare earth elements La, Ce, Pr, Sm and Er. The specific surface area of this modified zinc aluminate carrier is 10-100 m²/g, the pore size range is 3 nm-30 nm, and the pore volume range is 0.1-0.7 g/mL.

This modified zinc aluminate carrier has the characteristics of low acidity, and high mechanical strength, etc., compared to traditional zinc aluminate carriers. It helps to improve the stability of the carrier and reduce its surface acidity in the preparation of dehydrogenation catalysts subsequently, avoiding the defect of acid cracking due to excessive B acid in traditional carriers.

This modified zinc aluminate carrier is prepared by an impregnation method, a precipitation method, a coprecipitation method or a hydrothermal synthesis method. Preferably, the modified zinc aluminate carrier is prepared by a precipitation method or a coprecipitation method, and the precipitant used is at least one of ammonia water, sodium hydroxide, potassium hydroxide, sodium carbonate and urea; preferably, the modified zinc aluminate carrier is prepared by the gel sol method, and the gelling agent used is at least one of citric acid, nitric acid and hydrochloric acid.

This catalyst can be further applied to a fixed bed, a moving bed or a fluidized bed, with a reaction temperature of 550-620° C., preferably 570-610° C., and more preferably 580-600° C.; a reaction pressure of 10-150 kPa, preferably 20-100 kPa, and more preferably 30-70 kPa; a reaction space velocity of 0.1-2 h⁻¹, preferably 0.3-1.5 h⁻¹, and more preferably 0.5-1.0 h⁻¹.

The specific embodiments of the present invention are described in detail below. It should be understood that the specific embodiments described herein are only used to illustrate and explain the present invention, and are not used to limit the present invention.

### Example 1

This example provides a light alkane dehydrogenation catalyst, a preparation method of which includes:

weighing 377 g of aluminum nitrate, 187 g of zinc nitrate, and 4.33 g of lanthanum nitrate, and dissolving them in 1 L of deionized water for ultrasonic dissolution. Under high-speed stirring, dropwise adding ammonia water to adjust the pH to 5-8, then stirring the mixed solution at high speed to allow it to complete precipitation, followed by standing still and aging. Filtering and washing the aged product to obtain a filter cake, oven-drying same in an oven at 80° C. and calcining in a muffle furnace at 1000° C. to obtain the ZnLa_{0.01}Al_{1.99}O₄ carrier.

Weighing 10 g of the above carrier and dispersing it in 50 ml of deionized water, stirring the solution at high speed for 30 minutes to obtain a mixed solution A. Weighing the precursor solution of chloropalladic acid containing 0.1 g of Pd and 0.37 g of gallium nitrate into a beaker, to which adding 20 ml of deionized water for ultrasonic dissolution to obtain solution B. Under the condition of stirring the mixed solution A at high speed, dropwise adding solution B, and mixing evenly, stirring at room temperature for 2 hours, and then evaporating the solvent to dryness with a rotary evaporator. Oven-drying the obtained dry product in an oven at 80° C. and calcining it at 600° C. for 4 hours.

### Example 2

This example provides a light alkane dehydrogenation catalyst, a preparation method of which includes:

weighing 373 g of aluminium nitrate (213, 1.75 Mol), 181 g of zinc acetate (189, 0.95 Mol), and 3.26 g of cerium nitrate (326, 0.01 mol), and dissolving them in 1 L of deionized water for ultrasonic dissolution. Under high-speed stirring, dropwise adding ammonia water to adjust the pH to 5-8, then stirring the mixed solution at high speed to allow it to complete precipitation, followed by standing still and aging. Filtering and washing the aged product to obtain a filter cake, oven-drying same in an oven at 80° C. and calcining in a muffle furnace at 1000° C. to obtain the ZnCe_{0.01}Al_{1.99}O₄ carrier.

Weighing 10 g of the above carrier and dispersing it in 50 ml of deionized water, stirring the solution at high speed for 30 minutes to obtain a mixed solution A. Weighing the precursor solution of chloroplatinic acid containing 0.1 g of Pt and 0.37 g of gallium nitrate into a beaker, to which adding 20 ml of deionized water for ultrasonic dissolution to obtain solution B. Under the condition of stirring the mixed solution A at high speed, dropwise adding solution B, and mixing evenly, stirring at room temperature for 2 hours, and then evaporating the solvent to dryness with a rotary evaporator. Oven-drying the obtained dry product in an oven at 80° C. and calcining it at 600° C. for 4 hours.

### Example 3

This example provides a light alkane dehydrogenation catalyst, a preparation method of which includes:

weighing 133 g of aluminium chloride (213, 0.62 Mol), 181 g of zinc acetate (189, 0.95 Mol), and 2.9 g of praseodymium oxalate (545, 0.005), and dissolving them in 1 L of deionized water for ultrasonic dissolution. Under high-speed stirring, dropwise adding ammonia water to adjust the pH to 5-8, then stirring the mixed solution at high speed to allow it to complete precipitation, followed by standing still and aging. Filtering and washing the aged product to obtain a filter cake, oven-drying same in an oven at 80° C. and calcining in a muffle furnace at 1000° C. to obtain the ZnPr_{0.1}Al_{1.9}O₄ carrier.

Weighing 10 g of the above carrier and dispersing it in 50 ml of deionized water, stirring the solution at high speed for 30 minutes to obtain a mixed solution A. Weighing the precursor solution of chloroplatinic acid containing 0.1 g of Pt and 0.37 g of gallium nitrate into a beaker, to which adding 20 ml of deionized water for ultrasonic dissolution to obtain solution B. Under the condition of stirring the mixed solution A at high speed, dropwise adding solution B, and mixing evenly, stirring at room temperature for 2 hours, and then evaporating the solvent to dryness with a rotary evaporator. Oven-drying the obtained dry product in an oven at 80° C. and calcining it at 600° C. for 4 hours.

### Example 4

This example provides a light alkane dehydrogenation catalyst, a preparation method of which includes:

weighing 373 g of aluminum nitrate, 146 g of zinc acetate, and 86.6 g of lanthanum nitrate, and dissolving them in 1 L of deionized water for ultrasonic dissolution. Under high-speed stirring, dropwise adding sodium hydroxide to adjust the pH to 5-8, then stirring the mixed solution at high speed to allow it to complete precipitation, followed by standing still and aging. Filtering and washing the aged product to obtain a filter cake, oven-drying same in an oven at 80° C. and calcining in a muffle furnace at 1000° C. to obtain the ZnLa_{0.2}Al_{1.8}O₄ carrier.

Weighing 10 g of the above carrier and dispersing it in 50 ml of deionized water, stirring the solution at high speed for 30 minutes to obtain a mixed solution A. Weighing the precursor solution of chloroplatinic acid containing 0.1 g of Pt and 0.56 g of manganese acetate into a beaker, to which adding 20 ml of deionized water for ultrasonic dissolution to obtain solution B. Under the condition of stirring the mixed solution A at high speed, dropwise adding solution B, and mixing evenly, stirring at room temperature for 2 hours, and then evaporating the solvent to dryness with a rotary evaporator. Oven-drying the obtained dry product in an oven at 80° C. and calcining it at 600° C. for 4 hours.

### Example 5

This example provides a light alkane dehydrogenation catalyst, a preparation method of which includes:

weighing 133 g of aluminium chloride, 181 g of zinc acetate, and 5.48 g of cerium nitrate, and dissolving them in 1 L of deionized water for ultrasonic dissolution. Under high-speed stirring, dropwise adding ammonia water to adjust the pH to 5-8, then stirring the mixed solution at high speed to allow it to complete precipitation, followed by standing still and aging. Filtering and washing the aged product to obtain a filter cake, oven-drying same in an oven at 80° C. and calcining in a muffle furnace at 1000° C. to obtain the ZnCe_{0.01}Al_{1.99}O₄ carrier.

Weighing 10 g of the above carrier and dispersing it in 50 ml of deionized water, stirring the solution at high speed for 30 minutes to obtain a mixed solution A. Weighing the precursor solution of chloropalladic acid containing 0.1 g of Pd and 0.37 g of vanadium pentoxide into a beaker, to which adding 20 ml of deionized water for ultrasonic dissolution to obtain solution B. Under the condition of stirring the mixed solution A at high speed, dropwise adding solution B, and mixing evenly, stirring at room temperature for 2 hours, and then evaporating the solvent to dryness with a rotary evaporator. Oven-drying the obtained dry product in an oven at 80° C. and calcining it at 600° C. for 4 hours.

### Example 6

This example provides a light alkane dehydrogenation catalyst, a preparation method of which includes:

weighing 373 g of aluminum nitrate, 181 g of zinc acetate, and 4.33 g of lanthanum nitrate, and dissolving them in 1 L of deionized water for ultrasonic dissolution. Under high-speed stirring, dropwise adding sodium hydroxide to adjust the pH to 5-8, then stirring the mixed solution at high speed to allow it to complete precipitation, followed by standing still and aging. Filtering and washing the aged product to obtain a filter cake, oven-drying same in an oven at 80° C. and calcining in a muffle furnace at 1000° C. to obtain the ZnLa_{0.01}Al_{1.99}O₄ carrier.

Weighing 10 g of the above carrier and dispersing it in 50 ml of deionized water, stirring the solution at high speed for 30 minutes to obtain a mixed solution A. Weighing the precursor solution of rhodium chloride containing 0.1 g of Ru and 0.37 g of gallium nitrate into a beaker, to which adding 20 ml of deionized water for ultrasonic dissolution to obtain solution B. Under the condition of stirring the mixed solution A at high speed, dropwise adding solution B, and mixing evenly, stirring at room temperature for 2 hours, and then evaporating the solvent to dryness with a rotary evaporator. Oven-drying the obtained dry product in an oven at 80° C. and calcining it at 600° C. for 4 hours.

### Example 7

This example provides a light alkane dehydrogenation catalyst, a preparation method of which includes:

weighing 373 g of aluminum nitrate, 181 g of zinc acetate, and 4.33 g of lanthanum nitrate, and dissolving them in 1 L of deionized water for ultrasonic dissolution. Dropwise adding the dissolved metal solution and ammonia water together into the precipitation tank using a feed pump, controlling the pH of the solution to 5-8, then stirring the mixed solution at high speed to allow it to uniform precipitation, followed by standing still and aging. Filtering and washing the aged product to obtain a filter cake, oven-drying same in an oven at 80° C. and calcining in a muffle furnace at 1000° C. to obtain the ZnLa_{0.01}Al_{1.99}O₄ carrier.

Weighing 10 g of the above carrier and dispersing it in 50 ml of deionized water, stirring the solution at high speed for 30 minutes to obtain a mixed solution A. Weighing the precursor solution of chloroplatinic acid containing 0.1 g of Pt and 0.37 g of indium nitrate into a beaker, to which adding 20 ml of deionized water for ultrasonic dissolution to obtain solution B. Under the condition of stirring the mixed solution A at high speed, dropwise adding solution B, and mixing evenly, stirring at room temperature for 2 hours, and then evaporating the solvent to dryness with a rotary evaporator. Oven-drying the obtained dry product in an oven at 80° C. and calcining it at 600° C. for 4 hours.

### Example 8

This example provides a light alkane dehydrogenation catalyst, a preparation method of which includes:

weighing 373 g of aluminum nitrate, 183 g of zinc acetate, and 4.33 g of lanthanum nitrate, and dissolving them in 1 L of deionized water for ultrasonic dissolution. Weighing 480 g of citric acid, adding it into the above solution under stirring at high speed, stirring the mixed solution at high speed for 2 hours, heating up to 80° C., and evaporating the solvent in the mixed solution to obtain gel, oven-drying same in an oven at 80° C., and calcining in a muffle furnace at 1000° C. to obtain the ZnLa_{0.01}Al_{1.99}O₄ carrier.

Weighing 10 g of the above carrier and dispersing it in 50 ml of deionized water, stirring the solution at high speed for 30 minutes to obtain a mixed solution A. Weighing the precursor solution of chloroplatinic acid containing 0.1 g of Pt and 0.37 g of gallium nitrate into a beaker, to which adding 20 ml of deionized water for ultrasonic dissolution to obtain solution B. Under the condition of stirring the mixed solution A at high speed, dropwise adding solution B, and mixing evenly, stirring at room temperature for 2 hours, and then evaporating the solvent to dryness with a rotary evaporator. Oven-drying the obtained dry product in an oven at 80° C. and calcining it at 600° C. for 4 hours.

### Example 9

This example provides a light alkane dehydrogenation catalyst, a preparation method of which includes:

weighing 373 g of aluminum nitrate, 183 g of zinc acetate, and 4.33 g of lanthanum nitrate, and dissolving them in 1 L of deionized water for ultrasonic dissolution. Weighing 90 g of nitric acid, dropwise adding it into the above solution under stirring at high speed, stirring the mixed solution at high speed for 2 hours, heating up to 80° C., and evaporating the solvent in the mixed solution to obtain gel, oven-drying same in an oven at 80° C., and calcining in a muffle furnace at 1000° C. to obtain the ZnLa_{0.01}Al_{1.99}O₄ carrier.

Weighing 10 g of the above carrier and dispersing it in 50 ml of deionized water, stirring the solution at high speed for 30 minutes to obtain a mixed solution A. Weighing the precursor solution of chloroplatinic acid containing 0.1 g of Pt and 0.37 g of gallium nitrate into a beaker, to which adding 20 ml of deionized water for ultrasonic dissolution to obtain solution B. Under the condition of stirring the mixed solution A at high speed, dropwise adding solution B, and mixing evenly, stirring at room temperature for 2 hours, and then evaporating the solvent to dryness with a rotary evaporator. Oven-drying the obtained dry product in an oven at 80° C. and calcining it at 600° C. for 4 hours.

### Example 10

This example provides a light alkane dehydrogenation catalyst, a preparation method of which includes:

weighing 373 g of aluminum nitrate, 183 g of zinc acetate, and 4.33 g of lanthanum nitrate, and dissolving them in 1 L of deionized water for ultrasonic dissolution. Under high-speed stirring, dropwise adding sodium hydroxide to adjust the pH to 5-8, then stirring the mixed solution at high speed to allow it to complete precipitation, followed by standing still and aging. Filtering and washing the aged product to obtain a filter cake, oven-drying same in an oven at 80° C. and calcining in a muffle furnace at 1000° C. to obtain the ZnLa_{0.01}Al_{1.99}O₄ carrier.

Weighing 10 g of the above carrier and dispersing it in 50 ml of deionized water, stirring the solution at high speed for 30 minutes to obtain a mixed solution A. Weighing the precursor solution of chloroplatinic acid containing 0.1 g of Pt and 0.16 g of stannous chloride into a beaker, to which adding 20 ml of deionized water for ultrasonic dissolution to obtain solution B. Under the condition of stirring the mixed solution A at high speed, dropwise adding solution B, and mixing evenly, stirring at room temperature for 2 hours, and then evaporating the solvent to dryness with a rotary evaporator. Oven-drying the obtained dry product in an oven at 80° C. and calcining it at 600° C. for 4 hours.

### Example 11

This example provides a light alkane dehydrogenation catalyst, a preparation method of which includes:

weighing 373 g of aluminum nitrate, 181 g of zinc acetate, and 4.33 g of lanthanum nitrate, and dissolving them in 1 L of deionized water for ultrasonic dissolution. Under high-speed stirring, dropwise adding ammonia water to adjust the pH to 5-8, then stirring the mixed solution at high speed to allow it to complete precipitation, followed by standing still and aging. Filtering and washing the aged product to obtain a filter cake, oven-drying same in an oven at 80° C. and calcining in a muffle furnace at 1000° C. to obtain the ZnLa_{0.01}Al_{1.99}O₄ carrier.

Weighing 10 g of the above carrier and dispersing it in 50 ml of deionized water, stirring the solution at high speed for 30 minutes to obtain a mixed solution A. Weighing the precursor solution of platinum nitrate containing 0.1 g of Pt and 0.16 g of stannous chloride into a beaker, to which adding 20 ml of deionized water for ultrasonic dissolution to obtain solution B. Under the condition of stirring the mixed solution A at high speed, dropwise adding solution B, and mixing evenly, stirring at room temperature for 2 hours, and then evaporating the solvent to dryness with a rotary evaporator. Oven-drying the obtained dry product in an oven at 80° C. and calcining it at 600° C. for 4 hours.

### Comparative Example 1

This comparative example provides a light alkane dehydrogenation catalyst, the preparation method of which includes:

Weighing 10 g of commercial alpha-phase alumina and dispersing it into 50 ml of deionized water, stirring the solution at high speed for 30 minutes to obtain a mixed solution A. Weighing the precursor solution of chloroplatinic acid containing 0.1 g of Pt, to which adding 20 ml of deionized water for ultrasonic dissolution to obtain solution B. Under the condition of stirring the mixed solution A at high speed, dropwise adding solution B, and mixing evenly, stirring at room temperature for 2 hours, and then evaporating the solvent to dryness with a rotary evaporator. Oven-drying the obtained dry product in an oven at 80° C. and calcining it at 600° C. for 4 hours.

### Comparative Example 2

This comparative example provides a light alkane dehydrogenation catalyst, the preparation method of which includes:

weighing 373 g of aluminium nitrate, and dissolving it in 1 L of deionized water for ultrasonic dissolution. Under high-speed stirring, dropwise adding ammonia water to adjust the pH to 5-8, then stirring the mixed solution at high speed to allow it to complete precipitation, followed by standing still and aging. Filtering and washing the aged product to obtain a filter cake, oven-drying same in an oven at 80° C. and calcining in a muffle furnace at 1000° C. to obtain the Al₂O₃ carrier.

Weighing 10 g of the above carrier and dispersing it in 50 ml of deionized water, stirring the solution at high speed for 30 minutes to obtain a mixed solution A. Weighing the precursor solution of platinum nitrate containing 0.1 g of Pt and 0.37 g of gallium nitrate into a beaker, to which adding 20 ml of deionized water for ultrasonic dissolution to obtain solution B. Under the condition of stirring the mixed solution A at high speed, dropwise adding solution B, and mixing evenly, stirring at room temperature for 2 hours, and then evaporating the solvent to dryness with a rotary evaporator. Oven-drying the obtained dry product in an oven at 80° C. and calcining it at 600° C. for 4 hours.

### Comparative Example 3

This comparative example provides a light alkane dehydrogenation catalyst, the preparation method of which includes:

weighing 373 g of aluminium nitrate, and dissolving it in 1 L of deionized water for ultrasonic dissolution. Weighing 40 g of nitric acid, dropwise adding it into the above solution under stirring at high speed, stirring the mixed solution at high speed for 2 hours, heating up to 80° C., and evaporating the solvent in the mixed solution to obtain gel, oven-drying same in an oven at 80° C., and calcining in a muffle furnace at 1000° C. to obtain the Al₂O₃ carrier.

Weighing 10 g of the above carrier and dispersing it in 50 ml of deionized water, stirring the solution at high speed for 30 minutes to obtain a mixed solution A. Weighing the precursor solution of chloroplatinic acid containing 0.1 g of Pt and 0.37 of gallium nitrate into a beaker, to which adding 20 ml of deionized water for ultrasonic dissolution to obtain solution B. Under the condition of stirring the mixed solution A at high speed, dropwise adding solution B, and mixing evenly, stirring at room temperature for 2 hours, and then evaporating the solvent to dryness with a rotary evaporator. Oven-drying the obtained dry product in an oven at 80° C. and calcining it at 600° C. for 4 hours.

### Comparative Example 4

This comparative example provides a light alkane dehydrogenation catalyst, the preparation method of which includes:

Weighing 102 g of alumina and 65 g of zinc oxide into a beaker, to which adding 100 g of deionized water, and stirring same at high speed for 30 minutes. Weighing the precursor solution of chloroplatinic acid containing 0.1 g of Pt into a beaker, to which adding 20 ml of deionized water for ultrasonic dissolution to obtain solution B. Under the condition of stirring the mixed solution A at high speed, dropwise adding solution B, and mixing evenly, stirring at room temperature for 2 hours, and then evaporating the solvent to dryness with a rotary evaporator. Oven-drying the obtained dry product in an oven at 80° C. and calcining it at 600° C. for 4 hours.

### Comparative Example 5

This example provides a light alkane dehydrogenation catalyst, a preparation method of which includes:

weighing 373 g of aluminum nitrate, 181 g of zinc acetate, and 4.33 g of lanthanum nitrate, and dissolving them in 1 L of deionized water for ultrasonic dissolution. Under high-speed stirring, dropwise adding ammonia water to adjust the pH to 5-8, then stirring the mixed solution at high speed to allow it to complete precipitation, followed by standing still and aging. Filtering and washing the aged product to obtain a filter cake, oven-drying same in an oven at 80° C. and calcining in a muffle furnace at 1000° C. to obtain the ZnLa_{0.01}Al_{1.99}O₄ carrier.

Weighing 10 g of the above carrier and dispersing it in 50 ml of deionized water, stirring the solution at high speed for 30 minutes to obtain a mixed solution A. Weighing the precursor solution of platinum nitrate containing 0.1 g of Pt into a beaker, to which adding 20 ml of deionized water for ultrasonic dissolution to obtain solution B. Under the condition of stirring the mixed solution A at high speed, dropwise adding solution B, and mixing evenly, stirring at room temperature for 2 hours, and then evaporating the solvent to dryness with a rotary evaporator. Oven-drying the obtained dry product in an oven at 80° C. and calcining it at 600° C. for 4 hours.

The reaction conditions of carriers in Examples and Comparative Examples are summarized, as shown in Table 1:

TABLE 1

| | Active | | | | | | |
|---|---|---|---|---|---|---|---|
| | component | | Preparation | Precipitant/ | | | Rare earth |
| | precursor | Promoter | method | Gelling agent | Zn source | Al source | precursor |
| Example 1 | Chloropalladic | Gallium | Precipitation | Ammonia | Zinc | Aluminum | Lanthanum |
| | Acid | nitrate | method | water | nitrate | nitrate | nitrate |
| Example 2 | Chloroplatinic | Gallium | Precipitation | Ammonia | Zinc | Aluminum | Cerium |
| | Acid | nitrate | method | water | acetate | nitrate | nitrate |
| Example 3 | Chloroplatinic | Gallium | Precipitation | Ammonia | Zinc | Aluminium | Praseodymium |
| | Acid | nitrate | method | water | acetate | chloride | oxalate |
| Example 4 | Chloroplatinic | Manganese | Precipitation | Ammonia | Zinc | Aluminum | Lanthanum |
| | Acid | acetate | method | water | acetate | nitrate | nitrate |
| Example 5 | Chloropalladic | Gallium | Precipitation | Ammonia | Zinc | Aluminum | Vanadium |
| | Acid | nitrate | method | water | acetate | nitrate | pentoxide |
| Example 6 | Rhodium | Gallium | Precipitation | Sodium | Zinc | Aluminum | Lanthanum |
| | chloride | nitrate | method | hydroxide | acetate | nitrate | nitrate |
| Example 7 | Chloroplatinic | Indium | Coprecipitation | Sodium | Zinc | Aluminum | Lanthanum |
| | Acid | nitrate | method | hydroxide | acetate | nitrate | nitrate |
| Example 8 | Chloroplatinic | Gallium | Sol-gel | Citric acid | Zinc | Aluminum | Lanthanum |
| | Acid | nitrate | | | acetate | nitrate | nitrate |
| Example 9 | Chloroplatinic | Gallium | Sol-gel | Nitric acid | Zinc | Aluminum | Lanthanum |
| | Acid | nitrate | | | acetate | nitrate | nitrate |
| Example 10 | Chloroplatinic | Stannous | Precipitation | Sodium | Zinc | Aluminum | Lanthanum |
| | Acid | chloride | method | hydroxide | acetate | nitrate | nitrate |
| Example 11 | Platinum | Stannous | Precipitation | Sodium | Zinc | Aluminum | Lanthanum |
| | nitrate | chloride | method | hydroxide | acetate | nitrate | nitrate |
| Comparative | Chloroplatinic | Gallium | Impregnation | Commercial alumina | | | |
| Example 1 | Acid | nitrate | method | | | | |
| Comparative | Chloroplatinic | Gallium | Precipitation | Ammonia | / | Aluminum | |
| Example 2 | Acid | nitrate | method | water | | nitrate | |
| Comparative | Chloroplatinic | Gallium | Sol gel | | / | Aluminum | |
| Example 3 | Acid | nitrate | method | | | nitrate | |
| Comparative | Chloroplatinic | / | Impregnation | | | Alumina | Zinc oxide |
| Example 4 | Acid | | method | | | | |
| Comparative | Chloroplatinic | / | Precipitation | Sodium | Zinc | Aluminum | Lanthanum |
| Example 5 | Acid | | method | hydroxide | acetate | nitrate | nitrate |

To further illustrate the performance of the catalyst provided by the present invention, the following experiments are conducted.

### I. Propane Dehydrogenation Test

The adopted process flow is the existing process flow, which will not be elaborated in detail in the examples. The control parameters in the process flow are as follows: propane space velocity was 1 h⁻¹, an appropriate amount of hydrogen gas was introduced, the partial pressure of propane was maintained at 50 kPa, and the total pressure of the reaction system was atmospheric pressure; the bed temperature was 550-600° C. Among them, the preparation of carriers and catalyst compositions in examples and comparative examples are shown in Table 1, and the test results are shown in Table 2.

TABLE 2

| | | Propane | Propene | Propylene | By |
|---|---|---|---|---|---|
| | Catalyst identification | conversion rate % | selectivity % | yield % | product % |
| Example 1 | PtdGa/ZnLa_{0.01}Al_{1.99}O₄ | 37.45 | 89.14 | 33.39 | 4.07 |
| Example 2 | PtGa/ZnCe_{0.01}Al_{1.99}O₄ | 37.84 | 88.91 | 33.64 | 4.20 |
| Example 3 | PtGa/ZnPr_{0.01}Al_{1.99}O₄ | 37.08 | 89.19 | 33.07 | 4.01 |
| Example 4 | PtMn/ZnLa_{0.2}Al_{1.8}O₄ | 36.67 | 88.98 | 32.63 | 4.04 |
| Example 5 | PdGa/ZnCe_{0.01}Al_{1.99}O₄ | 38.23 | 88.78 | 33.94 | 4.29 |
| Example 6 | RuGaNa/ZnLa_{0.01}Al_{1.99}O₄ | 39.42 | 88.81 | 35.01 | 4.41 |
| Example 7 | PtIn/ZnLa_{0.01}Al_{1.99}O₄ | 41.54 | 89.22 | 37.07 | 4.48 |
| Example 8 | PtGa/ZnLa_{0.01}Al_{1.99}O₄ | 41.66 | 89.46 | 37.27 | 4.39 |
| Example 9 | PtGa/ZnLa_{0.01}Al_{1.99}O₄ | 40.88 | 88.52 | 36.18 | 4.69 |
| Example 10 | PtSnNa/ZnLa_{0.01}Al_{1.99}O₄ | 44.04 | 84.93 | 37.41 | 6.64 |
| Example 11 | PtSnNa/ZnLa_{0.01}Al_{1.99}O₄ | 42.93 | 85.87 | 36.86 | 6.07 |
| Comparative | Pt/Al₂0₃ | 21.20 | 80.12 | 16.99 | 4.22 |
| Example 1 | | | | | |
| Comparative | PtGa/Al₂0₃ | 26.08 | 84.25 | 21.97 | 4.11 |
| Example 2 | | | | | |
| Comparative | PtGa/Al₂0₃ | 28.04 | 85.15 | 23.88 | 4.16 |
| Example 3 | | | | | |
| Comparative | Pt/ZnAl₂04 | 27.93 | 84.25 | 23.53 | 4.40 |
| Example 4 | | | | | |
| Comparative | Pt/ZnLa_{0.01}Al_{1.99}O₄ | 34.55 | 87.03 | 30.07 | 4.48 |
| example 5 | | | | | |

As shown in Table 2, the conversion rate, selectivity, and propylene yield of the catalysts provided in Examples 1-11 of the this application for propane dehydrogenation are superior to those of Comparative Examples 1-4, thereby indicating that such a modified zinc aluminate carrier provided in this application has stronger stability and lower surface acidity of carrier compared to traditional zinc aluminate carriers, alumina carriers, and commercial alpha-phase alumina carriers, thereby avoiding the problem of acid cracking due to excessive B acid in the traditional carriers and significantly improving both the catalytic performance and stability of the catalysts. The catalytic performance of the catalysts provided in Examples 1-11 of this application is also superior to that of Comparative Example 5, indicating that the introduction of a promoter also compensates for some high-energy defect positions on the carrier to some extent, ensuring the comprehensive performance of the dehydrogenation catalyst.

Finally, it should be noted that the above is only preferred examples of the present invention and is not intended to limit the scope of protection of the present invention. Any modifications, equivalent substitutions, improvements, etc. made within the spirit and principles of the present invention should be included in the scope of protection of the present invention.

1. A light alkane dehydrogenation catalyst, wherein the catalyst uses at least one of precious metals Pt, Pd, Ru and Rh as an active component, at least one of transition metals Ga, V, In, Sn, Mn, Fe and Ni as a promoter, and modified zinc aluminate as a carrier;
the modified zinc aluminate carrier has a chemical composition of the general formula ZnMₓAl_{y}O₄, where x is 0.01-0.99, y is 0.01-1.99, and it satisfies x+y=2; M is selected from at least one of the rare earth elements La, Ce, Pr, Sm and Er.

2. The light alkane dehydrogenation catalyst according to claim 1, wherein based on the total mass of the catalyst on a dry basis, the content by mass percentage of the active component is 1-40 wt %, the content by mass percentage of the promoter is 1-20 wt %, with the balance being the modified zinc aluminate carrier.

3. The light alkane dehydrogenation catalyst according to claim 1, wherein the specific surface area of the modified zinc aluminate carrier is 10-100 m²/g, the pore size range is 3 nm-30 nm, and the pore volume range is 0.1-0.7 g/mL.

4. The light alkane dehydrogenation catalyst according to claim 1, wherein a precursor of the rare earth element is one or more of nitrate of the rare earth element, rare earth metal oxide, rare earth metal sulfate and rare earth metal organic acid salt.

5. The light alkane dehydrogenation catalyst according to claim 1, wherein a precursor of the precious metal is selected from one or more of metal halides, metal nitrates and metal complexes; and preferably, a precursor of the transition metal is one or more of oxides, inorganic salts or complexes of a metal element.

6. The light alkane dehydrogenation catalyst according to claim 1, wherein the modified zinc aluminate carrier is prepared by a gel sol method, an impregnation method, a precipitation method, a coprecipitation method or a hydrothermal synthesis method.

7. The light alkane dehydrogenation catalyst according to claim 6, wherein the modified zinc aluminate carrier is prepared by a precipitation method or a coprecipitation method, and the precipitant used is at least one of ammonia water, sodium hydroxide, potassium hydroxide, sodium carbonate and urea;
preferably, the modified zinc aluminate carrier is prepared by the gel sol method, and the gelling agent used is at least one of citric acid, nitric acid and hydrochloric acid.

8. A preparation method of the light alkane dehydrogenation catalyst according to claim 1, comprising:
under stirring conditions, adding a solution containing the active component and the promoter dropwise to a dispersion containing the modified zinc aluminate carrier, stirring the mixture for 1-3 hours, recovering the solvent, oven-drying and then calcining.

9. The preparation method of the light alkane dehydrogenation catalyst according to claim 8, wherein the temperature during the calcining process is 500-700° C. and the time is 3-5 hours.

10. Application of the light alkane dehydrogenation catalyst according to claim 1, wherein the catalyst is used for propane dehydrogenation, isobutane dehydrogenation, or propane/isobutane mixed gas dehydrogenation, and the catalyst is applied to a fixed bed, a moving bed or a fluidized bed, with a reaction temperature of 550-620° C., a reaction pressure of 10-150 kPa, and a reaction space velocity of 0.1-2 h⁻¹.
